(19) United States
(12) Patent Application Publication
(10) Pub. No.: US 2022/0002658 A1
(43) Pub. Date: Jan. 6, 2022

(54) CENTRIFUGAL MEMBER AND WASHING METHOD

(71) Applicant: TERUMO KABUSHIKI KAISHA, Tokyo (JP)

(72) Inventor: YUSUKE NOZAKI, Kanagawa (JP)

(21) Appl. No.: 17/479,831

(22) Filed: Sep. 20, 2021

Related U.S. Application Data
(63) Continuation of application No. PCT/JP2020/012735, filed on Mar. 23, 2020.

(30) Foreign Application Priority Data
Mar. 26, 2019 (JP) 2019-057924

(51) Int. Cl. C12M 1/00; C12N 5/0789; B04B 5/04

(52) U.S. Cl. CPC C12M 45/05; B04B 2005/0485; B04B 5/0442; C12N 5/0647

(57) ABSTRACT

Provided is a centrifugal member that centrifuges a cell suspension, the centrifugal member including: a cylindrical main body extending in a cylindrical shape in a centrifugal direction, having an inner end wall formed at an inner end in the centrifugal direction, and having an outer end in the centrifugal direction opened; a gasket slidably inserted into an inner portion of the cylindrical main body in the centrifugal direction; a pusher connected to an outer side of the gasket in the centrifugal direction; and a port provided on the inner end wall and communicating with the inner portion of the cylindrical main body.

### CROSS REFERENCE TO RELATED APPLICATIONS

The present application is a continuation of and claims benefit to PCT Application No. PCT/JP2020/012735 filed on Mar. 23, 2020, entitled “CENTRIFUGE MEMBER AND CLEANING METHOD” which claims priority to Japanese Patent Application No. 2019-057924 filed on Mar. 26, 2019. The entire disclosures of the applications listed above are hereby incorporated herein by reference, in their entirety, for all that they teach and for all purposes.

### FIELD

The present disclosure relates to a centrifugal member of a cell suspension used for a cell therapy and a washing method.

### BACKGROUND

Hematopoietic stem cell transplantation has been performed for a treatment of leukemia, malignant lymphoma, multiple myeloma, and the like. In the hematopoietic stem cell transplantation, a cell suspension containing hematopoietic stem cells, such as cord blood, bone marrow liquid, or peripheral blood, is cryopreserved, and the cell suspension is thawed and administered to a patient immediately before the transplantation.

The cell suspension is cryopreserved in a state of being enclosed in a flexible resin medical container (storage bag). Japanese Patent Application Nos. JP 2010-229256 A and JP 2014-180326 A disclose a resin medical container that can be used for storing a cell suspension.

### SUMMARY

In order to protect a cell membrane of a stem cell during the cryopreservation, about 10% of a protective agent composed of a bipolar solvent such as dimethyl sulfoxide (DMSO) is added to the cell suspension. By adding such a protective agent, growth of ice crystal at the time of freezing is suppressed, and destruction of the stem cell can be prevented.

However, the DMSO remaining in the cell suspension is of concern for its effect on health. In order to remove the DMSO from the cell suspension, an operation of centrifuging cells, and dispersing the cells in a harmless liquid agent again to dilute and suspend the cells is required. The operation described above needs to be performed aseptically (e.g., without introducing the risk of infection to the patient), and is not currently performed since it is complicated. Therefore, after the cell suspension is thawed, the cell suspension is administered to the patient while containing the protective agent.

Accordingly, a centrifugal member and a washing method, in which the cell suspension is capable of being washed by a simple operation, are desired.

According to an aspect of the present disclosure, there is provided a centrifugal member that centrifuges a cell suspension, the centrifugal member including: a cylindrical main body extending in a cylindrical shape, having an inner end wall formed at an inner end in a centrifugal direction, and having an outer end in the centrifugal direction opened; a gasket slidably inserted into an inner portion of the cylindrical main body in the centrifugal direction; a pusher connected to an outer side of the gasket in the centrifugal direction; and a port provided on the inner end wall and in fluidic communication with the inner portion of the cylindrical main body.

According to another aspect of the following disclosure, there is provided a washing method for washing a cell suspension that contains cells to be transplanted and is cryopreserved, by using a centrifugal member including a cylindrical main body extending in a cylindrical shape, having an inner end wall formed at an inner end in a centrifugal direction, and having an outer end in the centrifugal direction opened, a gasket slidably inserted into an inner portion of the cylindrical main body in the centrifugal direction, a pusher connected to an outer side of the gasket in the centrifugal direction, and a port provided at the inner end wall and in fluidic communication with an inner portion of the cylindrical main body, the method including: an introduction step of introducing the cell suspension into the inner portion of the cylindrical main body via the port; a centrifugation step of applying a centrifugal force to the centrifugal member to precipitate the cells on the gasket; a concentration step of pressing the pusher toward the inner end wall to remove a supernatant of the cell suspension via the port; and a dilution step of introducing a substitution liquid into the inner portion of the cylindrical main body via the port.

According to the centrifugal member and the washing method from the viewpoint described above, the cell suspension can be washed by the simple operation thereby separating or removing the protective agent (e.g., DMSO, etc.) from the cell suspension. This operation may be performed prior to administering the cell suspension to the patient.

### BRIEF DESCRIPTION OF THE DRAWINGS

FIG. 1 is a schematic view of a cell suspension washing system according to an embodiment of the present disclosure;

FIG. 2 is a perspective view of a centrifugal member according to an embodiment of the present disclosure;

FIG. 3 is a schematic view illustrating an introduction step of connecting a storage bag to the centrifugal member of FIG. 2 to introduce a cell suspension into the centrifugal member in accordance with embodiments of the present disclosure;

FIG. 4 is a cross-sectional view illustrating a centrifuging step by the centrifugal member in FIG. 3 in accordance with embodiments of the present disclosure;

FIG. 5 is a cross-sectional view illustrating a centrifuging step by a centrifugal member according to a modified example of FIG. 4 in accordance with embodiments of the present disclosure;

FIG. 6 is a schematic view illustrating a concentration step of removing a supernatant of a cell suspension by connecting a waste liquid bag to the centrifugal member of FIG. 4 in accordance with embodiments of the present disclosure;

FIG. 7 is a schematic view illustrating a diluting step of performing diluting and suspending by connecting a substitution liquid bag and an empty bag to the centrifugal member of FIG. 6 in accordance with embodiments of the present disclosure; and

FIG. 8 is an enlarged sectional view illustrating movement of a substitution liquid in the centrifugal member in the diluting step of FIG. 7 in accordance with embodiments of the present disclosure.

### DETAILED DESCRIPTION

Hereinafter, embodiments of the present disclosure will be described in detail with reference to the accompanying drawings.

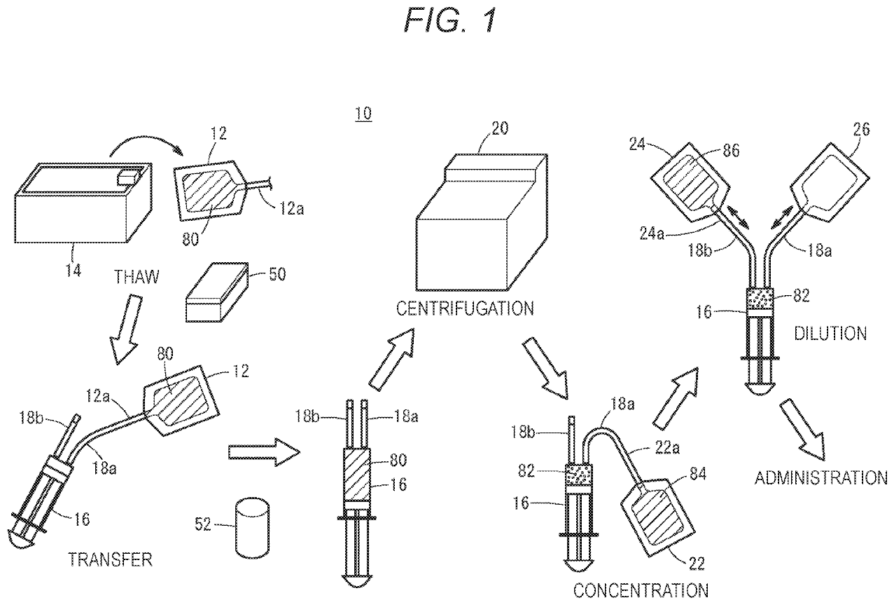

In some examples, washing of a cell suspension 80 may be performed by a washing system 10, as illustrated in FIG. 1. The washing system 10 may include a freezer 14, a centrifugal member 16, a centrifuge 20, an aseptic connecting device 50, and an aseptic sealing device 52. Among these, the freezer 14 stores a storage bag 12 storing the cell suspension 80 containing cells to be transplanted.

The cell suspension 80 contains, for example, hematopoietic stem cells as the cells to be transplanted. Note that the cells contained in the cell suspension 80 are not limited to the hematopoietic stem cells, and may be various cells such as neural stem cells, hepatic stem cells, skin stem cells, adipose stem cells, and/or germ stem cells. The cell suspension 80 is stored in a frozen state in the storage bag 12. In order to prevent the cells from being destroyed by the growth of ice crystals at the time of freezing, the cell suspension 80 contains a bipolar solvent such as DMSO as a protective agent at a concentration of about 10%.

The storage bag 12 is taken out of the freezer 14, and is used for a subsequent treatment after the cell suspension 80 is thawed under a condition of being heated to a room temperature or a predetermined temperature. The storage bag 12 includes a connection tube 12a made of a thermoplastic resin, and is connected to a connection tube 18a of the centrifugal member 16 by the aseptic connecting device 50 after the thawing. After the cell suspension 80 in the storage bag 12 is transferred to the centrifugal member 16, the storage bag 12 is aseptically separated from the centrifugal member 16 by the aseptic sealing device 52; the storage bag 12 is then discarded.

The aseptic connecting device 50 is a device that connects the connection tubes extending from another member (for example, the centrifugal member 16) to each other in an aseptic state, and is configured to connect the tubes containing a liquid or without a liquid in an aseptic state without exposing the tubes to an outside air. The aseptic connecting device 50 performs, for example, connection between the connection tube 12a of the storage bag 12 and the connection tube 18a of the centrifugal member 16. Furthermore, the aseptic sealing device 52 is configured to seal the connection tubes 12a and 18a in an aseptic state without exposing the connection tubes to the outside air (e.g., to air in an environment that is external to, or outside of, the connection tubes 12a and 18a)and then to cut the connection tubes.

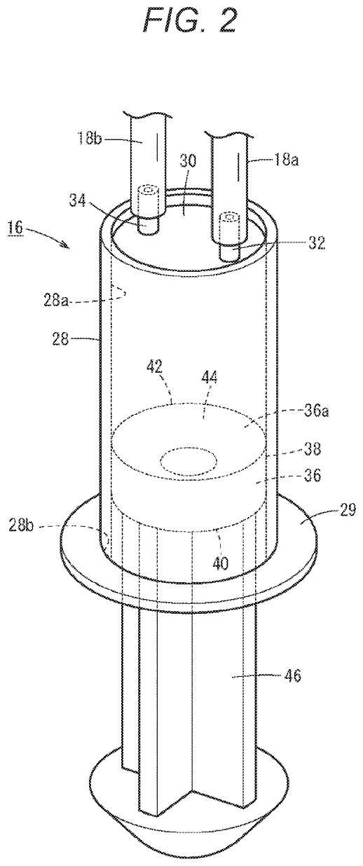

As illustrated in FIG. 2, the centrifugal member 16 may comprise a syringe-shaped member provided with a plurality of ports 32 and 34, that is configured to be capable of storing the cell suspension 80 in an inner portion 28a defined by a gasket 36. The centrifugal member 16 is attached to the centrifuge 20 in a state in which the ports 32 and 34 are directed toward a center of an action direction of a centrifugal force, and is configured to centrifuge the cell suspension 80 by applying the centrifugal force in a direction directed from the ports 32 and 34 to the gasket 36.

As illustrated in FIG. 2, the centrifugal member 16 may include a cylindrical main body 28 formed in a bottomed cylindrical shape. An inner end wall 30 is formed at one end (e.g., an inner end in the centrifugal direction) of the cylindrical main body 28, and an opening 28b is formed at the other end (e.g., an outer end in the centrifugal direction). A flange portion 29 is formed on an outer circumference of the opening 28b. A shape of the flange portion 29 can be appropriately set in accordance with a shape of an attachment portion of the centrifuge 20. The inner end wall 30 is formed in a wall shape so as to close an entire inner end of the cylindrical main body 28. A pair of the ports 32 and 34 are formed on the inner end wall 30.

The ports 32 and 34 are formed to protrude toward the inside of the inner end wall 30 in a cylindrical shape, and through holes communicating with the inner portion 28a of the cylindrical main body 28 are respectively formed at the center portions of the ports 32 and 34. The port 32 and the port 34 are disposed to be spaced apart from each other by a predetermined distance in a radial direction with the center of the inner end wall 30 interposed therebetween. The connection tube 18a is connected to the port 32, and a connection tube 18b is connected to the port 34.

The connection tubes 18a and 18b are formed of a thermoplastic resin so as to be connected or cut by the aseptic connecting device 50 and the aseptic sealing device 52. Lengths of the connection tubes 18a and 18b can be, for example, 5 cm or greater in order to facilitate handling of the aseptic connecting device 50 and the aseptic sealing device 52.

The gasket 36 is inserted into the inner portion 28a of the cylindrical main body 28. The gasket 36 is formed to have an inner diameter substantially equal to an inner diameter of the inner portion 28a of the cylindrical main body 28, and is configured to be slidable along a longitudinal direction of the cylindrical main body 28. Substantially equal may correspond to a diameter that matches the inner diameter of the inner portion 28a, is greater than the inner diameter of the inner portion 28a (e.g., in an uncompressed state) and that matches the inner diameter of the inner portion 28a in a compressed state, and/or a diameter that is less than the inner diameter of the inner portion 28a. In any event, a portion of the gasket 36 contacts a surface or portion of the inner portion 28a. The gasket 36 is made of an elastic material such as rubber or elastomer, and defines the inner portion 28a of the cylindrical main body 28 into a region on the inner end side (e.g., the side of the ports 32 and 34) and a region on the outer end side in an airtight and liquid-tight manner. An outer circumferential portion 38 of the gasket 36 is a sliding portion that moves in contact with the cylindrical main body 28.

Furthermore, on an inner end surface 36a of an inside of the gasket 36, a recess 44 is formed so as to be recessed outward in the centrifugal direction as being separated from a boundary portion 42 between the gasket 36 and the cylindrical main body 28. That is, in the gasket 36 of the embodiment, the recess 44 is formed to have a pyramid, conical, and/or a conical frustum shape in which the center portion is recessed most outward (e.g., in the axial, or centrifugal, direction). Note that, the shape of the recess 44 is not limited to the pyramid shape, and may be a U-shape in cross section, an arc-shape in cross section, a W-shape in cross section, or the like. The recess 44 is configured such that the cells in the cell suspension 80 are precipitated and collected when the centrifugal force is applied.

A pusher 46 is connected to an outer end surface 40 on the outside of the gasket 36. The pusher 46 is fixed to the gasket 36 by a method of fitting, adhesion, molding, or welding, and is configured such that the pusher 46 and the gasket 36 are integrally displaced. When the pusher 46 is pushed and moved inward, a volume of the inner portion 28a decreases, and when the pusher 46 is pulled out outward, the volume of the inner portion 28a increases.

As illustrated in FIG. 1, the centrifugal member 16 is set on a rotor of the centrifuge 20 and centrifugation is performed. The centrifuge 20 is configured to apply, for example, an acceleration of 100 G to 300 G (centrifugal force) to the centrifugal member 16 for a predetermined time.

The washing system 10 further includes a waste liquid bag 22, a substitution liquid bag 24, and an empty bag 26 for suspension as consumables. The waste liquid bag 22 is a bag that stores a supernatant 84 of the cell suspension 80 centrifuged in the centrifugal member 16. The waste liquid bag 22 is provided as an empty bag containing no content, and is configured to be connectable to (and in fluid communication with) the centrifugal member 16 via a connection tube 22a.

The substitution liquid bag 24 is a bag storing a substitution liquid 86 such as physiological saline or Ringer's solution, and is configured to be connectable to the centrifugal member 16 via a connection tube 24a. The empty bag 26 is connected to the centrifugal member 16 together with the substitution liquid bag 24, and is configured to urge suspension of the cell concentration portion 82 by allowing an inflow substitution liquid 86 to flow.

Next, a method for washing the cell suspension 80 using the centrifugal member 16 will be described together with the action of the centrifugal member 16.

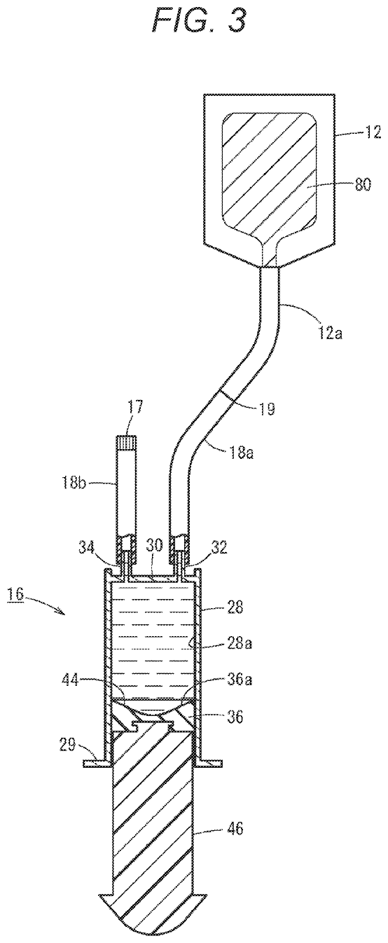

As illustrated in FIG. 3, the storage bag 12 after the thawing is connected to the centrifugal member 16. That is, the connection tube 12a of the storage bag 12 and one connection tube 18a of the centrifugal member 16 are connected to each other via a welding connection portion 19, or joint. The connection tube 12a and the connection tube 18a are connected by the aseptic connecting device 50 illustrated in FIG. 1. Note that a distal end portion of the other connection tube 18b of the centrifugal member 16 illustrated in FIG. 3 is a closed portion 17 welded in a crushed, clamped, sealed, or otherwise closed state.

Next, the pusher 46 of the centrifugal member 16 is pulled to transfer the cell suspension 80 stored in the storage bag 12 to the inner portion 28a of the centrifugal member 16. Stated another way, moving the pusher 46 in a direction away from the ports 32 and 34 of the centrifugal member 16, may create a vacuum, or negative pressure in the inner portion 28a of the centrifugal member 16. This negative pressure may cause the cell suspension 80 to move from the storage bag 12 to the inner portion 28a of the centrifugal member 16. After the transfer of the cell suspension 80 is completed, the connection tube 18a and the connection tube 12a are cut by using the aseptic sealing device 52, and the storage bag 12 is separated from the centrifugal member 16. The cut portion of the connection tube 18a is sealed using a closed portion 17 welded in a crushed state (e.g., sealing an inner portion of the connection tube 18a from an environment external to the connection tube 18a, etc.). The separated storage bag 12 may be discarded.

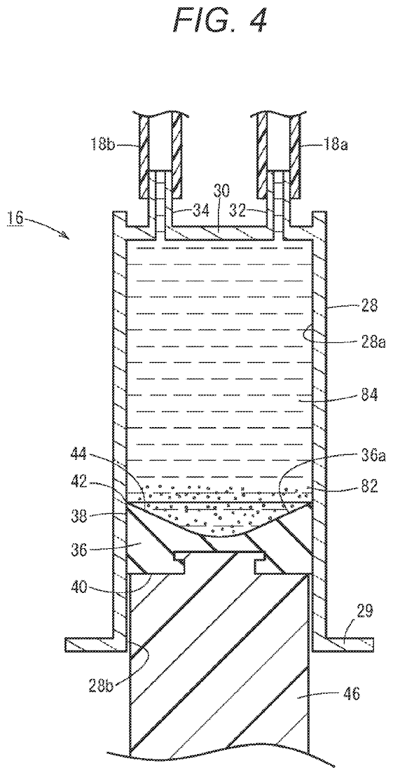

Next, as illustrated in FIG. 1, the centrifugal member 16 is set in the centrifuge 20, and the centrifugal force is applied to the centrifugal member 16. The centrifuge 20 causes acceleration due to centrifugal force to be applied in a direction from the ports 32 and 34 toward the gasket 36 in the centrifugal member 16 illustrated in FIG. 4. As a result, the cells having a relatively large specific gravity in the cell suspension 80 are precipitated on the gasket 36 side, and the cell suspension 80 is separated into the cell concentration portion 82 and the supernatant 84.

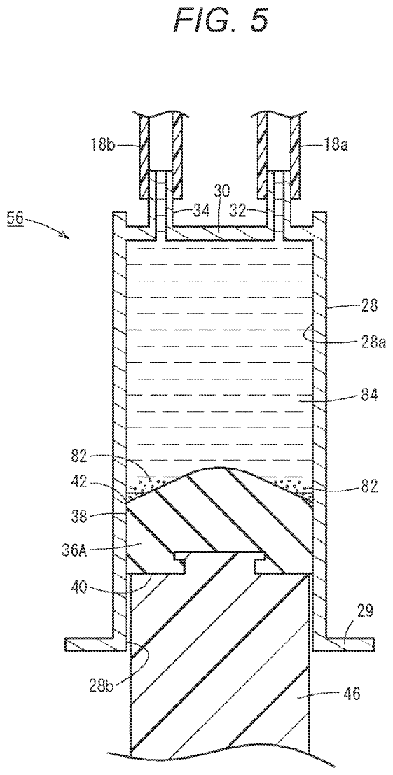

As illustrated at least in FIG. 4, the cells are accumulated in the recess 44 of the gasket 36 by the centrifugal force and precipitated in a portion away from the boundary portion 42 around the gasket 36. On the other hand, in a case of a gasket 36A of the centrifugal member 56 according to a modified example illustrated in FIG. 5, an end surface of the gasket 36A is formed to have a convex shape on a center side of the centrifugal direction. In a case of a centrifugal member 56, the cells are precipitated near the boundary portion 42 around the gasket 36A. When the gasket 36A of the modified example is advanced toward the ports 32 and 34, the cells may be sandwiched between the boundary portion 42 of the gasket 36A and the cylindrical main body 28. Therefore, the cells are damaged and the number of the effective cells decreases.

On the other hand, according to the centrifugal member 16 of the embodiment illustrated in FIG. 4, since most of the cells are precipitated at a place away from the boundary portion 42 of the gasket 36, the damage of the cells can be prevented, and the decrease in the number of the effective cells can be suppressed, even when the gasket 36 is advanced.

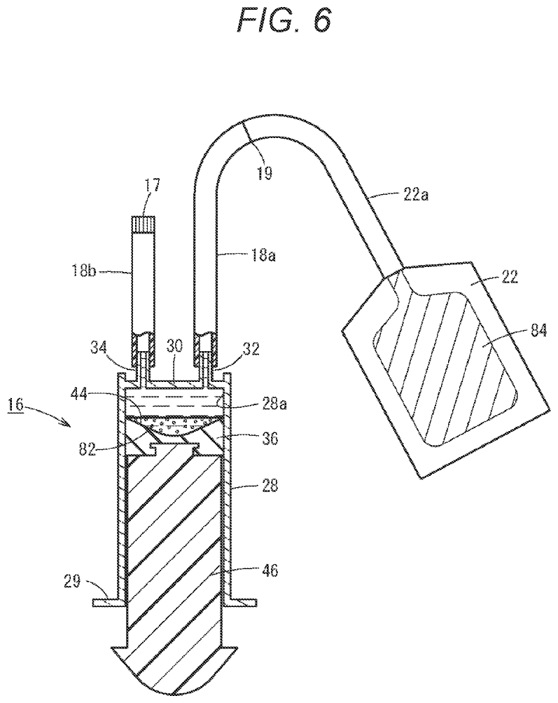

After that, the centrifugal member 16 is taken out from the centrifuge 20. Then, as illustrated in FIG. 6, the connection tube 22a of the waste liquid bag 22 is connected to the connection tube 18a of the centrifugal member 16. The connection tube 22a and the connection tube 18a are connected to each other by using the aseptic connecting device 50.

After that, the pusher 46 of the centrifugal member 16 is pressed toward the ports 32 and 34 to advance the gasket 36. According to this, the supernatant 84 obtained by centrifuging the cell suspension 80 is discharged from the inner portion 28a of the centrifugal member 16 and stored in the waste liquid bag 22. Most of the protective agent such as the DMSO is removed together with the supernatant 84 to be discharged to the waste liquid bag 22. The cell concentration portion 82 remains in the inner portion 28a of the centrifugal member 16, and the concentration step is completed. After that, the waste liquid bag 22 is separated from the centrifugal member 16.

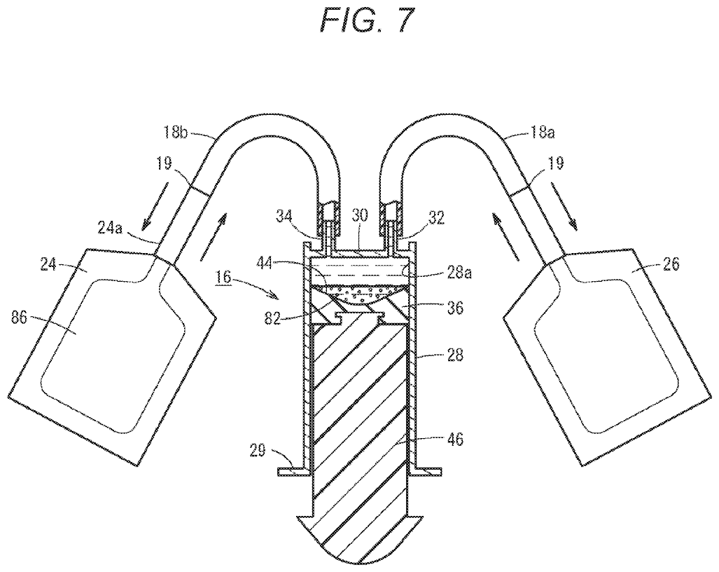

Next, as illustrated in FIG. 7, the empty bag 26 is connected to one port 32 of the centrifugal member 16 including the cell concentration portion 82 and the connection tube 18a, and the substitution liquid bag 24 is connected to the other port 34 and the connection tube 18b. Then, the substitution liquid bag 24 is pressed to send the substitution liquid 86 (for example, physiological saline, Ringer's solution, or the like) into the inner portion 28a of the centrifugal member 16.

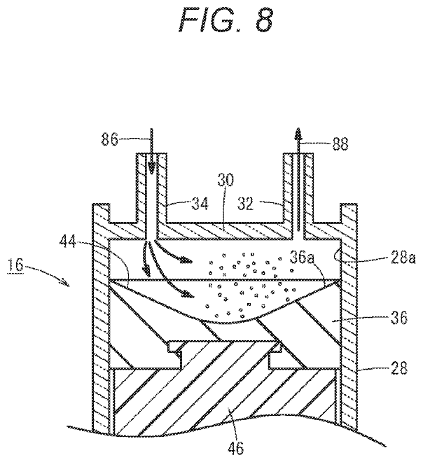

As illustrated in FIG. 8, in the inner portion 28a of the centrifugal member 16, the substitution liquid 86 flowing in from the port 34 flows toward the port 32 while raising the cells precipitated in the recess 44 as indicated by an arrow. The substitution liquid 86 flowing out of the centrifugal member 16 flows into the empty bag 26.

After that, a cell suspension 88 obtained by suspending the cells in the substitution liquid 86 is obtained by moving the substitution liquid 86 between the substitution liquid bag 24 and the empty bag 26 one or more times. Accordingly, the dilution step is completed. In the cell suspension 88 obtained in this manner, the protective agent such as the DMSO is diluted with the substitution liquid 86, and toxicity to a living body is reduced, if not eliminated.

Note that the cell suspension 88 may be further subjected to concentration by the centrifugation, and dilution and suspension by the substitution liquid 86 repeatedly one or more of times to further remove the protective agent, as desired.

The centrifugal member 16 of the embodiment and the method for washing the cell suspension 80 using the centrifugal member have one or more of the following effects and/or benefits detailed below.

The centrifugal member 16 of the embodiment, which centrifuges the cell suspension 80 containing the cells to be transplanted, includes the cylindrical main body 28 extending in a cylindrical shape in the centrifugal direction, having the inner end wall 30 formed at an inner end in the centrifugal direction, and having an outer end in the centrifugal direction opened, the gasket 36 slidably inserted into the inner portion 28a of the cylindrical main body 28 in the centrifugal direction, the pusher 46 connected to an outer side of the gasket 36 in the centrifugal direction, and the ports 32 and 34 provided at the inner end wall 30 and communicating with the inner portion 28a of the cylindrical main body 28. By using the centrifugal member 16, the supernatant 84 separated from the cell suspension 80 can be easily removed by operating the pusher 46 and the gasket 36.

In the centrifugal member 16 described above, the gasket 36 may have a recess 44 which is recessed outward in the centrifugal direction from the boundary portion 42 with the cylindrical main body 28, the recess 44 being formed on the inner end surface 36a on the inner side in the centrifugal direction. According to this, since most of the cells are precipitated in a portion away from the boundary portion 42, it is possible to prevent the cells from being damaged due to the advancement of the gasket 36.

In the centrifugal member 16 described above, the inner end surface 36a of the gasket 36 may have an inclined surface which is recessed outward as being separated from the boundary portion 42, the inclined surface being formed in the vicinity (e.g., within 1 millimeter (mm), within 1.1 mm, within 1.5 mm, within 2.0 mm, within 5.0 mm, etc.) of the boundary portion 42 with the cylindrical main body 28. According to this, the cells can be precipitated in a portion away from the boundary portion 42 by the action of the centrifugal force. In this case, in the centrifugal member 16, the recess 44 of the gasket 36 may be formed to have a pyramid shape such that the center portion of the gasket 36 is recessed most outward.

In the centrifugal member 16 described above, the inner end wall 30 of the cylindrical main body 28 may be provided with a plurality of the ports 32 and 34. According to this, when the concentrated cells are diluted with the substitution liquid 86, the substitution liquid 86 can be introduced into the inner portion 28a of the cylindrical main body 28 so as to raise the cells, and the cells can be efficiently suspended in the substitution liquid 86 without wasting the cells.

In the centrifugal member 16 described above, the flexible connection tubes 18a and 18b for connecting with an external bag may be connected to the ports 32 and 34. According to this, the external bag can be connected to the centrifugal member 16 while maintaining the aseptic state by using the aseptic connecting device 50 or the aseptic sealing device 52.

In the centrifugal member 16 described above, the connection tubes 18a and 18b may be made of a thermoplastic resin. According to this, the external bag can be connected to the centrifugal member 16 while maintaining the aseptic state by using the aseptic connecting device 50 or the aseptic sealing device 52.

The washing method of the embodiment is a method for washing the cell suspension 80 that contains the cells to be transplanted and is cryopreserved, by using the centrifugal member 16 including the cylindrical main body 28 extending in a cylindrical shape in the centrifugal direction, having the inner end wall 30 formed at an inner end in the centrifugal direction, and having an outer end in the centrifugal direction opened, the gasket 36 slidably inserted into the inner portion 28a of the cylindrical main body 28 in the centrifugal direction, the pusher 46 connected to an outer side of the gasket 36 in the centrifugal direction, and the ports 32 and 34 provided at the inner end wall 30 and communicating with the inner portion 28a of the cylindrical main body 28. The washing method includes an introduction step of introducing the cell suspension 80 into the inner portion 28a of the cylindrical main body 28 via the ports 32 and 34, a centrifugation step of applying the centrifugal force to the centrifugal member 16 to precipitate the cells on the gasket 36, a concentration step of pressing the pusher 46 toward the inner end wall 30 to remove the supernatant 84 of the cell suspension 80 via the port 32, and a dilution step of introducing the substitution liquid 86 into the inner portion 28a of the cylindrical main body 28 via the port 34. By the washing method, the protective agent contained in the cell suspension 80 can be removed by a simple operation.

In the above description, the centrifugal member and the washing method have been described with reference to the preferred embodiments, but the centrifugal member and the washing method are not limited to the embodiments described above, and it goes without saying that various modifications can be made without departing from the gist of the present disclosure.

What is claimed is:

1. A centrifugal member, comprising:
a cylindrical main body extending in a cylindrical shape, having an inner end wall formed at an inner end in a centrifugal direction, and having an outer end in the centrifugal direction opened;
a gasket slidably inserted into an inner portion of the cylindrical main body in the centrifugal direction;
a pusher connected to an outer side of the gasket in the centrifugal direction; and
at least one port provided on the inner end wall and in fluidic communication with the inner portion of the cylindrical main body.

2. The centrifugal member of claim 1, wherein the gasket has a recess which is recessed outward in the centrifugal direction from a boundary portion with the cylindrical main body, the recess being formed on an inner end surface on an inner side of the gasket in the centrifugal direction.

3. The centrifugal member of claim 2, wherein the inner end surface of the gasket has an inclined surface which is recessed outward in the centrifugal direction away from the boundary portion, the inclined surface being formed in a vicinity of the boundary portion with the cylindrical main body.

4. The centrifugal member of claim 2, wherein the recess of the gasket is formed to have a pyramid shape such that a center portion of the gasket is recessed most outward.

5. The centrifugal member of claim 1, wherein the at least one port comprises a plurality of ports provided on the inner end wall of the cylindrical main body.

6. The centrifugal member of claim 1, wherein a flexible tube is connected to the at least one port, the flexible tube configured to connect to an external bag.

7. The centrifugal member of claim 6, wherein the flexible tube comprises a thermoplastic resin.

8. A method for washing a cryopreserved cell suspension by using a centrifugal member including a cylindrical main body extending in a cylindrical shape, having an inner end wall formed at an inner end in a centrifugal direction, and having an outer end in the centrifugal direction opened, a gasket slidably inserted into an inner portion of the cylindrical main body in the centrifugal direction, a pusher connected to an outer side of the gasket in the centrifugal direction, and at least one port provided at the inner end wall and in fluidic communication with the inner portion of the cylindrical main body, the method comprising:
introducing the cell suspension into the inner portion of the cylindrical main body via the at least one port;
applying a centrifugal force to the centrifugal member to precipitate cells of the cell suspension on the gasket;
pressing the pusher toward the inner end wall to remove a supernatant of the cell suspension via the at least one port; and
introducing a substitution liquid into the inner portion of the cylindrical main body via the at least one port.

9. The method of claim 8, wherein the gasket comprises a recess disposed in an inner end surface on an inner side of the gasket in the centrifugal direction and extending from a boundary portion of the cylindrical main body toward the pusher in the centrifugal direction.

10. The method of claim 9, wherein the recess has a first diameter equal to a diameter of the inner portion of the cylindrical main body at the inner side of the gasket, and wherein the recess has a second diameter smaller than the first diameter at a first distance from the inner side of the gasket.

11. The method of claim 10, wherein the recess in a cross section view is one of a pyramid shape, a conical shape, a U-shape, an arc-shape, or a W-shape.

12. The method of claim 8, wherein the at least one port comprises a plurality of ports provided on the inner end wall of the cylindrical main body.

13. The method of claim 12, wherein the method further comprises:
attaching, to a first port of the plurality of ports, a first bag, the first bag comprising the substitution liquid;
attaching, to a second port of the plurality of ports, a second bag; and
causing the substitution liquid to flow from the first bag into the second bag, wherein the substitution liquid flows through the inner portion of the cylindrical main body.

14. An apparatus, comprising:
a cylindrical main body extending in a centrifugal direction from a closed end to an open end, the closed end comprising an end wall;
a gasket slidably inserted into an inner portion of the cylindrical main body, wherein a pusher connected to a first end of the gasket is configured to slide the gasket along the inner portion of the cylindrical main body in the centrifugal direction; and
at least one port disposed in the inner end wall, the at least one port in fluidic communication with the inner portion of the cylindrical main body.

15. The apparatus of claim 14, wherein the gasket further comprises a recess, the recess extending from a second end of the gasket toward the first end of the gasket in the centrifugal direction.

16. The apparatus of claim 15, wherein the recess has a first diameter at the second end of the gasket, wherein the recess has a second diameter at a first distance from the second end of the gasket, and wherein the first diameter is greater than the second diameter.

17. The apparatus of claim 16, wherein the recess in a cross section view is one of a pyramid shape, a conical shape, a U-shape, an arc-shape, or a W-shape.

18. The apparatus of claim 16, wherein the recess is at least partially formed from an inclined surface, and wherein the inclined surface extends from a boundary portion between the inner portion of the cylindrical main body and the gasket in the centrifugal direction toward the first end of the gasket.

19. The apparatus of claim 14, further comprising:
a flexible tube comprising a first end and a second end, wherein the first end of the flexible tube is connected to the at least one port, and wherein the second end of the flexible tube is connected to an external bag.

20. The apparatus of claim 19, wherein the flexible tube comprises a thermoplastic resin.
